(19) United States
(12) Patent Application Publication
(10) Pub. No.: US 2022/0359081 A1
(43) Pub. Date: Nov. 10, 2022

(54) SYSTEMS AND METHODS FOR DETERMINING NEONATAL MORTALITY IN ANIMALS

(71) Applicant: MARS, INCORPORATED, McLean, VA (US)

(72) Inventor: Achraf Adib-Lesaux, Aimargues (FR)

(73) Assignee: MARS, INCORPORATED, McLean, VA (US)

(21) Appl. No.: 17/621,837

(22) PCT Filed: Jun. 25, 2020
(86) PCT No.: PCT/US2020/039646

§ 371 Date: Dec. 22, 2021

Related U.S. Application Data
(60) Provisional application No. 62/866,225, filed on Jun. 25, 2019.

(51) Int. Cl. G16H 50/30

(52) U.S. Cl. CPC G16H 50/30

(57) ABSTRACT

Systems and methods for determining neonatal mortality in animals, and more specifically for determining risk factors and thresholds thereof indicative of neonatal mortality. Such systems can include a software application platform which provides a user with the ability to receive customized information relating to an animal's risk of neonatal mortality as displayed on a user interface. Specifically, a user can input data, for example, one or more animal specific biomarkers among other factors, and subsequently receive identification of a predicted risk of neonatal mortality and customized recommendations and/or intervention steps for the specific animal.

### CROSS-REFERENCE TO RELATED APPLICATIONS

This application claims priority to U.S. Patent Application Ser. No. 62/866,225, filed on Jun. 25, 2019, which is incorporated herein by reference in its entirety.

### 1. FIELD OF THE INVENTION

The present disclosure generally relates to systems and methods for determining neonatal mortality in animals, and more specifically for determining risk factors and thresholds thereof indicative of neonatal mortality. Such systems and methods can help a user, such as a veterinarian and/or breeder, evaluate and/or monitor one or more animal biomarkers, among other factors, to determine an estimated risk of neonatal mortality and perform an intervention.

### 2. BACKGROUND

Neonatal mortality rates in animals are used for both clinical and research purposes. For example, information regarding the risk factors and probability of neonatal mortality in specific types of animals and species can be helpful for the optimal management, monitoring, and treatment of such animals. Risk factors and thresholds thereof for neonatal mortality can be difficult to identify. In particular, with respect to kittens, the characterization of potential risk factors along with assessment of their value as predictors of neonatal mortality have not been adequately evaluated.

Thus, there remains a need for systems and methods for accurate and reliable identification of risk factors of neonatal mortality in animals, and more particularly, in the context of kittens. There further remains a need to provide standards—not just references—that assess the health and vitality of an animal based on such factors, as well as a need for diagnosing abnormalities after birth in order to reduce neonatal mortality rates. The use of biomarkers among other factors, which can indicate an animal's likelihood of mortality, would significantly enhance a veterinarian and/or breeder's ability to identify animals with a high risk and make informed treatment decisions.

### 3. SUMMARY OF THE INVENTION

The present disclosure generally relates to systems and methods for determining neonatal mortality in animals. More specifically, the present disclosure relates to systems and methods that allow a user, such as a veterinarian and/or breeder, to evaluate and/or monitor one or more biomarkers of an animal among other factors to determine an estimated risk of neonatal mortality. Such indications can be used as a predictive tool for neonatal mortality, and provide an increased ability of the user to identify animals with a high risk and make informed treatment decisions.

The present disclosure provides a method of diagnosing a risk of neonatal mortality in non-human animals. The method can include receiving one or more first biomarker inputs relating to a first animal. The method can also include comparing the one or more first biomarker inputs of the first animal to at least one predetermined reference biomarker input stored in a reference database in order to obtain relevant health trend information relating to the first animal. The predetermined reference biomarker input can include related biomarker inputs of normal, healthy animals of the same species. In addition, the method can include determining, based on the comparing, whether the first animal is at risk for at least one neonatal mortality indications. Further, the method can include providing a subject determined to be at risk for at least one neonatal mortality indications with a customized recommendation. The first biomarker input determined to be above or below the predetermined reference biomarker input indicates an increased likelihood of neonatal mortality in the first animal. The method can also include displaying the customized recommendation and the relevant health trend information of the first animal on a graphical user interface.

The present disclosure also provides a computer system for diagnosing a risk of neonatal mortality in non-human animals. The system can include a processor and a memory storing instructions that, when executed by the processor, cause the computer system to receive one or more first biomarker inputs relating to a first animal, and compare the one or more first biomarker inputs of the first animal to at least one predetermined reference biomarker input stored in a reference database in order to obtain relevant health trend information relating to the first animal. The predetermined reference biomarker input can include related biomarker inputs of healthy animals of the same species as the first animal. The computer system can also be caused to determine, based on the comparing, whether the first animal is at risk for at least one neonatal mortality indications, and provide a subject determined to be at risk for at least one neonatal mortality indications with a customized recommendation. The first biomarker input determined to be above or below the predetermined reference biomarker input indicates an increased likelihood of neonatal mortality in the first animal. In addition, the computer system can also be caused to display the customized recommendation and the relevant health trend information of the first animal on a graphical user interface.

In certain embodiments, the customized recommendation can be an intervention step for correction of the at least one neonatal mortality indications.

In certain embodiments, the one or more first biomarker inputs of the first animal can include animal breed, birth weight, litter size, litter heterogeneity, queening season, and early growth rate.

In certain embodiments, the at least one neonatal mortality indication includes low birth weight, low early growth rate, or combinations thereof

### 4. BRIEF DESCRIPTION OF THE DRAWINGS

FIG. 1 illustrates a computer system configured for providing a website having a neonatal mortality application, according to certain non-limiting embodiments described herein;

FIG. 2 illustrates a more detailed view of a server of FIG. 1, according to one embodiment described herein;

FIG. 3 illustrates a user computing system used to access a web site and utilize the neonatal mortality application, according to certain non-limiting embodiments described herein;

FIG. 4 illustrates a conceptual diagram of applying a neonatal mortality application display scheme to a user interface, according to certain non-limiting embodiments described herein;

FIGS. 5A and 5B illustrate birth weight results per breed according to certain non-limiting embodiments as provided in Example 1. FIG. 5A provides a graph illustrating birth weight distribution per breed. FIG. 5B provides a graph illustrating the effect on birth weight by breed;

FIG. 6 illustrates a graph providing a relationship between maternal weight and newborn birth weight according to certain non-limiting embodiments as provided in Example 1;

FIG. 7 illustrates a flow diagram providing for the determination of practical thresholds to identify at risk kittens for neonatal mortality according to certain non-limiting embodiments as provided in Example 1; and

FIGS. 8A-8C illustrate results of birth weight and neonatal mortality in kittens according to certain non-limiting embodiments as provided in Example 1. FIG. 8A illustrates a graph providing the relationship between birth weight and 0-2 months neonatal mortality in kittens. FIG. 8B illustrates a graph providing the relationship between birth weight and early growth rate with respect to 2 days-2 months neonatal mortality in kittens. FIG. 8C illustrates a graph providing the relationship between birth weight and early growth rate with respect to 2 days-2 months neonatal mortality in kittens.

### 5. DETAILED DESCRIPTION

In certain non-limiting embodiments, the present disclosure generally relates to systems and methods for determining neonatal mortality in animals. More specifically, the present disclosure relates to systems and methods that help a user, such as a veterinarian and/or breeder, to evaluate and/or monitor one or more biomarkers of an animal among other factors to determine an estimated risk of neonatal mortality. Such determination can be used, for example, to identify at-risk animals and make informed treatment decisions or interventions.

For clarity and not by way of limitation, this detailed description is divided into the following sub-portions:

5.1. Definitions;

5.2 Biomarkers; and

5.3. Systems and methods for determining neonatal mortality in animals.

### 5.1. Definitions

The terms used in this specification generally have their ordinary meanings in the art, within the context of this disclosure and in the specific context where each term is used. Certain terms are discussed below, or elsewhere in the specification, to provide additional guidance in describing the compositions and methods of the disclosure and how to make and use them.

As used herein, the words “a” or “an,” when used in conjunction with the term “comprising” in the claims and/or the specification, can mean “one,” but they are also consistent with the meaning of “one or more,” “at least one,” and/or “one or more than one.” Furthermore, the terms “having,” “including,” “containing” and “comprising” are interchangeable, and one of skill in the art will recognize that these terms are open ended terms. The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value.

Alternatively, particularly with respect to systems or processes, the term can mean within an order of magnitude, preferably within 5-fold, and more preferably within 2-fold, of a value.

As used herein, the term “animal” refers to animals including, but not limited to, cats and the like. Domestic cats are particular non-limiting examples of animals.

As used herein, the term “biomarker” can refer to a characteristic that is objectively measured and evaluated as an indicator of physiological biological processes, pathogenic processes, or pharmacologic responses to a therapeutic intervention. In some non-limiting embodiments, the term “biomarker” can refer to any substance, structure, or process that can be measured in the body or its products and influence or predict the incidence of outcome or disease. In other non-limiting embodiments, the term “biomarker” can refer to an anthropometric measurement.

As used herein, the term “cattery” refers to a location where cats are commercially housed.

As used herein, the terms “comprises,” “comprising,” or any other variation thereof, are intended to cover a non-exclusive inclusion, such that a process, method, article, or apparatus that comprises a list of elements does not include only those elements but can include other elements not expressly listed or inherent to such process, method, article, or apparatus.

The term “feline” as used herein refers to a cat or of or relating to cats or to the biological family Felidae. For example, the term “feline” as used herein can refer to domestic cats.

The terms “kitten” or “kittens” as used herein refers to a juvenile cat or cats.

The term “litter” as used herein refers to offspring at one birth of an animal.

The term “neonatal” as used herein refers to newborn animals, such as newborn kittens. The terms “neonatal death” or “neonatal mortality” as used herein refer to the death or the state of being subject to death at the neonatal stage of an animal.

The term “reference database” as used herein refers to the set of references, charts, data points, graphs, media, code, data bits, and information for animals of specific sex, breed, and/or size, for one or more measurable factors.

The terms “sequential” or “sequentially” as used herein means that information is input in a successive manner such that a first portion of information is input at a first time, a second portion of information is input at a second time subsequent to the first time, and so on. The time between sequential inputs can be, for example, one or several days, weeks, months, or the like.

The term “user” as used herein includes, for example, a person or entity that owns a computing device or wireless device; a person or entity that operates or utilizes a computing device or a wireless device; or a person or entity that is otherwise associated with a computing device or wireless device. For example, in certain non-limiting embodiments a user can be a veterinarian, breeder, caregiver, or owner. It is contemplated that the term “user” is not intended to be limiting and can include various examples beyond those described.

The term “image” as used herein includes, for example, messages, photos, videos, blogs, advertisements, notifications, and any other type of media which can be visually consumed by a user. It is contemplated that the term “image” is not intended to be limiting and can include various examples beyond those described.

The terms “tomcat” or “tomcats” as used herein refers to a male cat or cats.

The terms “queen” or “queens” as used herein refers to an unspayed female cat or cats.

The term “queening” as used herein refers to the act of a cat giving birth or delivering a kitten or kittens.

### 5.2. Biomarkers

In certain non-limiting embodiments, one or more biomarker inputs related to a first animal can be received. The one or more first biomarker inputs related to the first animal can then be compared to at least one predetermined reference biomarker input stored in a reference database to obtain relevant health trend information relating to the first animal. The predetermined reference biomarker input, for example, can include related biomarker inputs of normal, healthy animals of a same species.

For example, the biomarker can be a breed or a species. Examples of the biomarker can include, but are not limited to, canine or feline breed, breed group, weight at birth, litter size, litter heterogeneity, season of queening, and early growth rate. Additional non-limiting examples of biomarkers include sex, growth rate of litter, type of delivery (caesarian), gestation length (time), age of parents, weight, length, body temperature, body mass index, total body fat distribution, caloric intake, spaying or neutering status, total body water, body cell mass, heart rate, blood pressure, and/or arterial stiffness. In certain non-limiting embodiments, the level of the biomarkers in the animal can be detected and quantified by any means known in the art.

In certain non-limiting embodiments, the breeds and/or breed groups can indicate an increased risk for neonatal mortality. In other non-limiting embodiments, the breeds and/or breed groups can indicate a decreased risk for neonatal mortality. For example, the cat breed Abyssinian/Somali, Balinese/Mandarin/Oriental/Siamese, Bengal, Birman, British, Chartreux, Egyptian Mau, Maine Coon, Norwegian Forest, Persian/Exotic, Ragdoll, Russian Blue/Nebelung, Siberian can indicate an increased risk for neonatal mortality. In other examples, the cat breed Abyssinian/Somali, Balinese/Mandarin/Oriental/Siamese, Bengal, Birman, British, Chartreux, Egyptian Mau, Maine Coon, Norwegian Forest, Persian/Exotic, Ragdoll, Russian Blue/Nebelung, Siberian can indicate a decreased risk for neonatal mortality.

In some-non limiting embodiments, the weight at birth can indicate an increased risk for neonatal mortality. In other non-limiting embodiments, the weight at birth can indicate a decreased risk for neonatal mortality. The risk for neonatal mortality can be determined by comparison to a predetermined reference value based on average body weight at birth in a population of dogs or cats in the dataset. The risk for neonatal mortality, for example, can be determined by comparison to a predetermined reference value based on average body weight at birth in a breed of dogs or cats in the dataset. In certain embodiments, a lower level of body weight at birth compared to a predetermined reference value based on average levels of body weight at birth in a control population indicates an increased risk of neonatal mortality. In certain non-limiting embodiments, the average levels of body weight at birth in the dataset population can be between about 70 g and about 350 g, between about 80 g and about 340 g, between about 90 g and about 330 g, or between about 100 g and about 320 g. In certain embodiments, the average levels of body weight at birth in a reference breed can be between about 70 g and about 350 g.

In certain non-limiting embodiments, the litter size can indicate an increased risk for neonatal mortality or a decreased risk for neonatal mortality. The term “litter size” can refer to the total number of litters born alive. In some non-limiting embodiments, the litter heterogeneity can indicate an increased risk or decreased risk for neonatal mortality. In some other non-limiting embodiments, early growth rate can indicate an increased or decreased risk for neonatal mortality. In addition, or as an alternative, the season of queening can indicate an increased risk or decreased risk for neonatal mortality. In certain non-limiting embodiments, the biomarker can be a protein, a metabolite, or a nucleic acid. Examples of biomarker can include, but are not limited to, insulin, pro-insulin, glucose, fasting free fatty acids, HDL-cholesterol, LDL-cholesterol, C-peptide, adiponectin, peptide YY, hemoglobin A1C, interleukins, cytokines, chemokines, albumin, myosin, calcium, phosphate, magnesium, bilirubin, parathyroid hormone, progesterone, and relaxin. Additional non-limiting examples of biomarkers include allelic variants, polymorphisms, single nucleotide polymorphisms, exosomes, miRNAs, long noncoding RNAs, fibrinogen, hemoglobin, and insulin-like growth factors. In certain non-limiting embodiments, the biomarker can be evaluated in the litter, in the queen during the gestational period, and/or in the parents.

One or more biomarkers can indicate an increased risk or a decreased risk for neonatal mortality. Litters with low birth rate can be at risk of neonatal mortality. Litters with low birth weight and low growth rate can also be at risk for neonatal mortality.

The ranges of average levels for the biomarkers can account for 50 to 100% of the healthy, normal population. For some biomarkers, the ranges of average levels for the biomarkers can account for 80 to 95%. Therefore, about 5-25% of the population can have values above the higher end of an average/normal range, and about another 5-25% of the population can have values below the low end of an average/normal range. In certain embodiments, the ranges and validity of the biomarkers can be determined by each laboratory or testing, depending on the machine and/or on the population of dogs or cats tested to determine an average/normal range. Additionally, laboratory tests can be impacted by sample handling and machine maintenance/calibration. Updates to machines can also result in changes in the normal ranges. Any one of these factors can be considered for adjusting the average levels and/or the predetermined reference values of each biomarker.

### 5.3. Systems and Methods for Determining Neonatal Mortality in Animals

In certain non-limiting embodiments, systems and methods for determining neonatal mortality in animals are provided. In certain non-limiting embodiments, the system can be a computing system including a neonatal mortality application server which can be accessed by a user's computer.

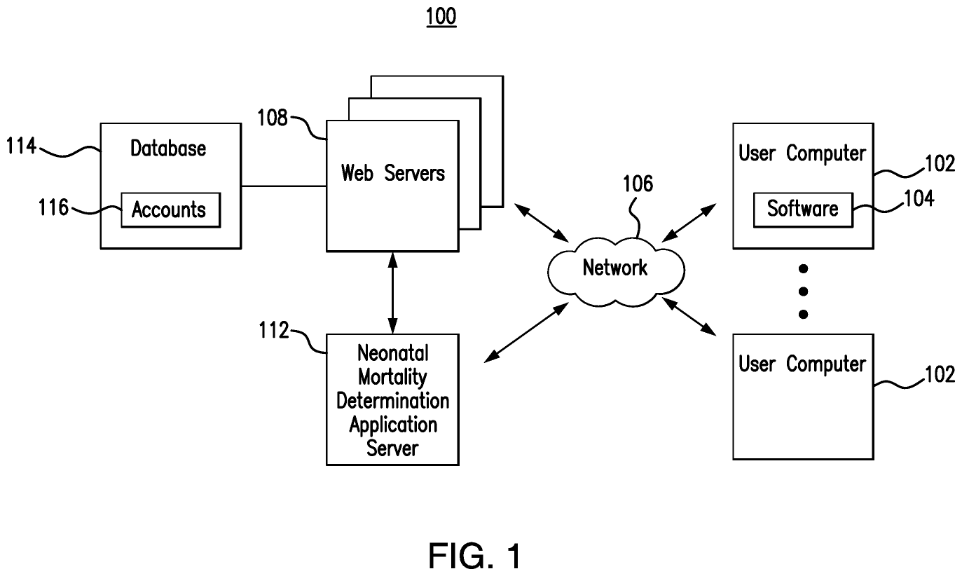

FIG. 1 illustrates a computing system 100 configured for providing a neonatal mortality application in which embodiments of the disclosure can be practiced. As shown, the computing system 100 can include a plurality of web servers 108, a neonatal mortality application server 112, and a plurality of user computers (for example, mobile/wireless devices) 102 (only two of which are shown for clarity), each of which can be connected to a communications network 106 (for example, the Internet). The web servers 108 can communicate with the database 114 via a local connection (for example, a Storage Area Network (SAN) or Network Attached Storage (NAS)) over the Internet (for example, a cloud based storage service). The web servers 108 can be configured to either directly access data included in the database 114 or can be configured to interface with a database manager that can be configured to manage data included with the database 114. An account 116 is a data object that can store data associated with a user, such as the user's email address, password, contact information, billing information, animal information, and the like.

Each user computer 102 can include conventional components of a computing device, for example, a processor, system memory, a hard disk drive, a battery, input devices such as a mouse and a keyboard, and/or output devices such as a monitor or graphical user interface, and/or a combination input/output device such as a touchscreen which not only can receive input but also can display output. Each web server 108 and the neonatal mortality application server 112 can include a processor and a system memory (not shown), and can be configured to manage content stored in database 114 using, for example, relational database software and/or a file system. Web servers 108 can be programmed to communicate with one another, user computer 102, and the neonatal mortality application server 112 using a network protocol such as, for example, the TCP/IP protocol. The neonatal mortality application server 112 can communicate directly with the user computer 102, for example, through the communications network 106. The user computer 102 can be programmed to execute software 104, such as web browser programs and other software applications, and can access web pages and/or application managed by web servers 108, for example, by specifying a uniform resource locator (URL) that can direct to web servers 108.

In the embodiments described below, users can respectively operate the user computer 102 that can be connected to the web servers 108 over the communications network 106. Web pages can be displayed to a user via user computer 102. The web pages can be transmitted from the web servers 108 to the user's computer 102 and can be processed by the web browser program stored in that user's computer 102 for display through a display device and/or a graphical user interface in communication with the user's computer 102.

In one example, information and/or images displayed on the user's computer 102 can relate to animal health information via a graph or chart accessed via an online database. The user's computer 102 can access the animal health information via the communications network 106 which, in turn, retrieves the animal health information from the web servers 108 connected to the database 114 and causes the information and/or images to be displayed through a graphical user interface of the user's computer 102. The online information and/or images, and/or the neonatal mortality application, can be managed with a username and password combination, or other similar restricted access/verification required access method, which can allow the user to “log in” and access the information.

It is noted that the user computer 102 can be a personal computer, laptop, mobile computing device, smart phone, video game console, home digital media player, network-connected television, set top box, and/or other computing devices having components suitable for communicating with the communications network 106. The user computer 102 can also execute other software applications configured to receive animal neonatal mortality information from the neonatal mortality application, such as, but not limited to, text and/or image display software, media players, computer and video games, and/or widget platforms, among others.

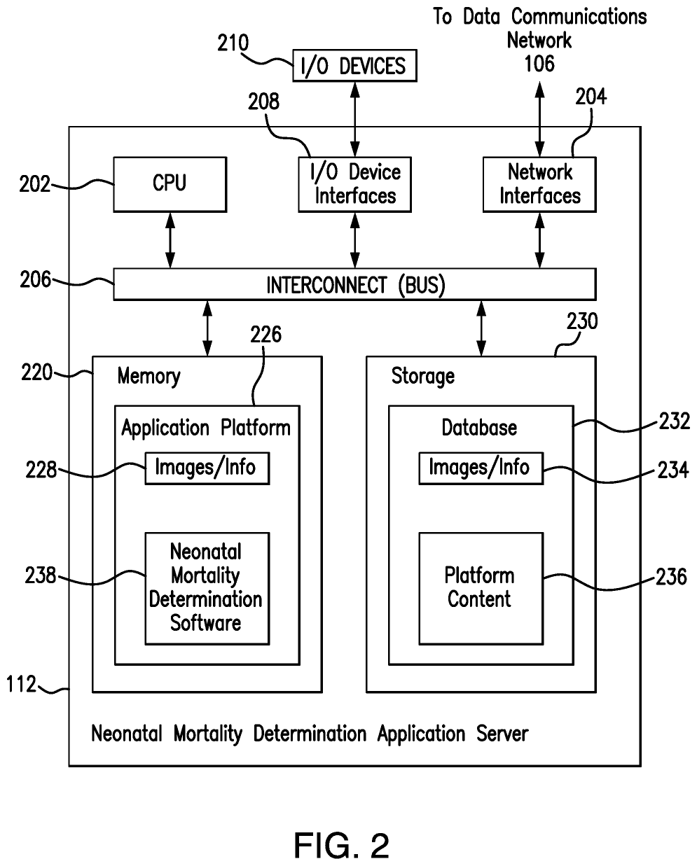

FIG. 2 illustrates a more detailed view of the neonatal mortality application server 112 of FIG. 1. The neonatal mortality application server 112 can include, without limitation, a central processing unit (CPU) 202, a network interface 204, memory 220, and storage 230 communicating via an interconnect 206. The neonatal mortality application server 112 can also include I/O device interfaces 208 connecting I/O devices 210 (for example, keyboard, video, mouse, audio, touchscreen, etc.). The neonatal mortality application server 112 can further include the network interface 204 configured to transmit data via data communications network 106.

CPU 202 can retrieve and execute programming instructions stored in the memory 220 and can generally control and coordinate operations of other system components. Similarly, the CPU 202 can store and retrieve application data residing in the memory 220. The CPU 202 can be included to be representative of a single CPU, multiple CPUs, a single CPU having multiple processing cores, and the like. The interconnect 206 can be used to transmit programming instructions and application data between CPU 202, I/O device interfaces 208, storage 230, network interfaces 204, and memory 220.

Memory 220 can be generally included to be representative of a random access memory and, in operation, stores software application and data for use by the CPU 202. Although shown as a single unit, the storage 230 can be a combination of fixed and/or removable storage devices, such as fixed disk drives, floppy disk drives, random access memory, hard disk drives, non-transitory computer-readable medium, flash memory storage drives, tape drives, removable memory cards, CD-ROM, DVD-ROM, Blu-Ray, HD-DVD, optical storage, network attached storage (NAS), cloud storage, or a storage area-network (SAN) configured to store non-volatile data.

Memory 220 can store instructions and logic for executing an application platform 226 which can include images 228 and/or neonatal mortality software 238. Storage 230 can store images and/or information 234 and other user generated media and can include a database 232 which can be configured to store images and/or information 234 associated with the application platform content 236. The database 232 can be any type of storage device. Database 232 can store application content relating to data associated with user generated media or images. In certain non-limiting embodiments, database 232 can store or include other application features for providing a user with an application platform that uses evidenced-based weight charts for animals, derived from biomarkers such as body weight and breed, among others. Database 232 can also include other biomarkers to create standards applicable to specific breeds within a species, and to recommend interventions or treatment for animal health.

Network computers are another type of computer system that can be used in conjunction with the disclosures provided herein. Network computers do not usually include a hard disk or other mass storage, and the executable programs can be loaded from a network connection into the memory 220 for execution by the CPU 202. A web TV system can be also considered to be a computer system, but it can lack some of the features shown in FIG. 2, such as certain input or output devices. A typical computer system will usually include at least a processor, memory, and an interconnect coupling the memory to the processor.

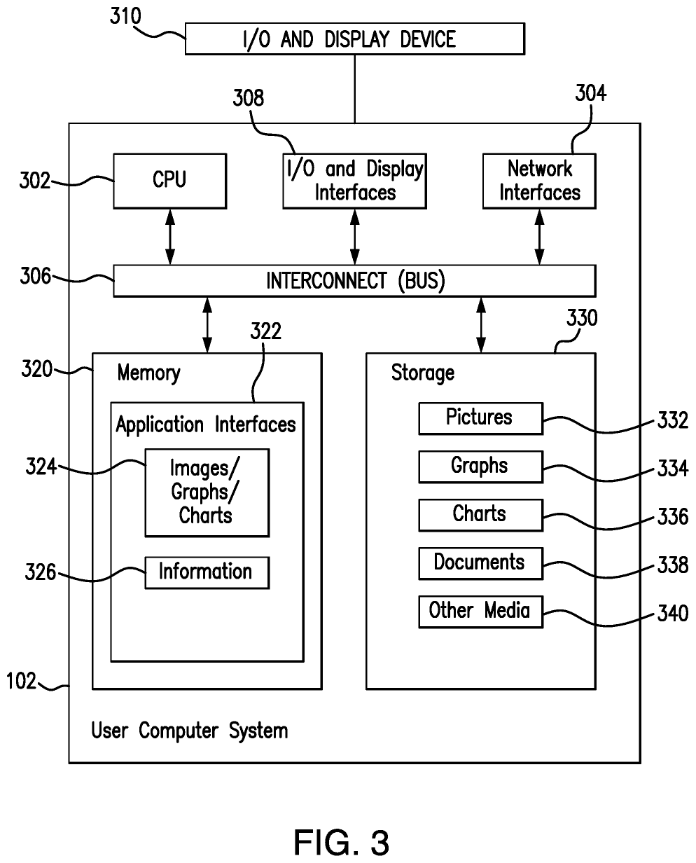

FIG. 3 illustrates a user computer 102 used to access the neonatal mortality application 112 and display images and/or information associated with the application platform 226. User computer 102, for example, can be a desktop computer, a laptop computer, a mobile device, or any other user equipment. User computer 102 can include, without limitation, a central processing unit (CPU) 302, a network interface 304, an interconnect 306, a memory 320, and storage 330. User computer 102 can also include an I/O device interface 308 connecting I/O devices 310 (for example, keyboard, display, touchscreen, and mouse devices) to the user computer 102.

Like CPU 202, CPU 302 can be included to be representative of a single CPU, multiple CPUs, a single CPU having multiple processing cores, etc., and the memory 320 can be generally included to be representative of a random access memory. Interconnect 306 can be used to transmit programming instructions application data between the CPU 302, I/O device interfaces 308, storage 330, network interface 304, and memory 320. Network interface 304 can be configured to transmit data via the communications network 106, for example, to stream or provide content from the neonatal mortality application server 112. Storage 330, such as a hard disk drive or solid-state storage drive (SSD), can store non-volatile data. Storage 330 can contain pictures 332, graphs 334, charts 336, documents 338, and other media 340. Illustratively, the memory 320 can include an application interface 322, which itself can display images 324, such as graphs or charts among others, and/or information 326. The application interface 322 can provide one or more software applications which can allow the user to access media items and other content hosted by the neonatal mortality application server 112.

All of the above terms are merely convenient labels applied to these physical quantities. Unless specifically stated otherwise as apparent from the following discussion, it is appreciated that throughout the description, discussions utilizing terms such as “processing” or “computing” or “calculating” or “determining” or “displaying” or “analyzing” or the like, refer to the action and processes of a computer system, server, or any other electronic computing device, that manipulates and transforms data represented as physical (electronic) quantities within the computer system's registers and memories into other data, similarly represented as physical quantities within the computer system memories, registers, or other such information storage, transmission, or display devices.

The present example also relates to an apparatus for performing the operations herein. This apparatus can be specially constructed for the required purposes, or it can comprise a general purpose computer selectively activated or reconfigured by a computer program stored in the computer. Such a computer program can be stored in a computer readable storage medium, such as, but is not limited to, read-only memories (ROMs), random access memories (RAMs), EPROMs, EEPROMs, flash memory, magnetic or optical cards, any type of disk including floppy disks, optical disks, CD-ROMs, and magnetic-optical disks, or any type of media suitable for storing electronic instructions, and each coupled to a computer system interconnect.

The algorithms and displays presented herein are not inherently related to any particular computer or other apparatus. Various general purpose systems can be used with programs in accordance with the teachings herein, or it can prove convenient to construct a more specialized apparatus to perform the required method operations. The structure for a variety of these systems will appear from the description above. In addition, the present examples are not described with reference to any particular programming language, and various examples can thus be implemented using a variety of programming languages.

As described in greater detail herein, certain non-limiting embodiments of the disclosure provide a computer system or mobile device, as shown in FIGS. 1 and 3, through which a user can receive customized information relating to an animal's health and/or neonatal mortality risks based on data input relating to a specific animal. The customized information can be displayed on a graphical user interface and/or a display device. Furthermore, the user can customize, via a selection of at least one biomarker, the information received, such as animal neonatal mortality risk or health information, displayed on a graphical user interface and/or display device, from which the computer system can apply and display relevant health information and/or an intervention recommendation.

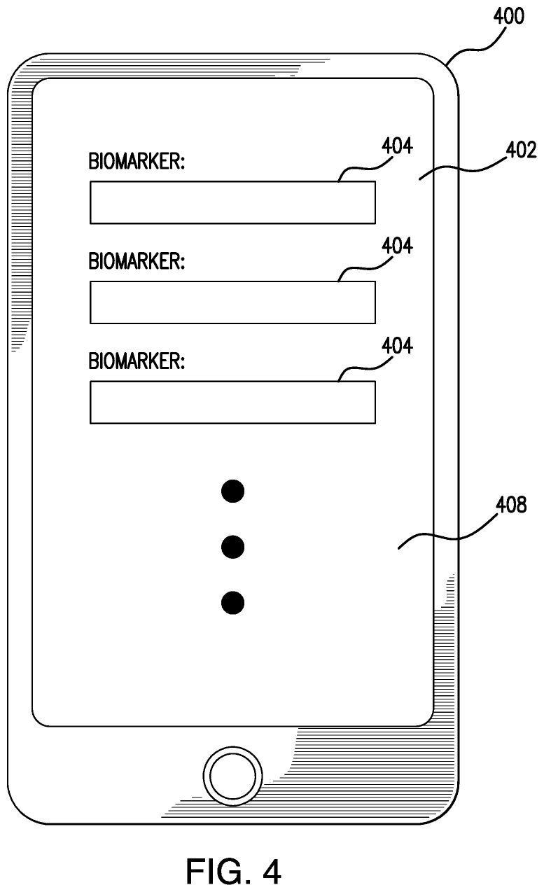

FIG. 4 is a conceptual diagram illustrating application of neonatal mortality application display schemes to a user interface 400, according to certain non-limiting embodiments described herein. The user interface 400 illustrated in FIG. 4 can be accessible via a web browser application (not illustrated) and include a plurality of web-based user interface elements, for example, a header, a footer, a body, borders, links, text blocks, graphics, images, media, charts, graphs, and the like, which can be arranged to present digital information, customized recommendations, and/or images on a web page within the web browser application. For example, user interface 400 can include a main window 402, that is configured to receive user input, such as one or more biomarkers 404, and/or display information, recommendation(s), and/or images contained within the web page based on user input.

By way of example only, and not intended to be limiting, in certain embodiments one type of biomarker input can be a cat breed. Cat breed classifications are well known in the art. Such cat breeds or breed groups can include, for example, Bengal, Birman, British, Chartreux, Egyptian Mau, Main Coon, Norwegian Forest Cat, Persian/Exotic, Ragdoll, Russian Blue/Nebelung, Siberian, and Abyssinian/Somali, Balinese/Mandarin/Oriental/Siamese. For example, a user can input “Maine Coon” for breed. If the breed of the cat is not known, no information can be entered by user. In another non-limiting embodiment, litter size (i.e., total number of kittens born alive), litter heterogeneity (e.g., within-litter variation of birth weight, expressed as the coefficient of variation CV, ratio of the standard deviation to the mean), queening season, early growth rate, cattery identification, queen identification, or combinations thereof can be used as factors or biomarkers. Early growth rate, for example, can be calculated using the following equation:

$$\frac{weight at 2 days - weight at birth}{weight at birth} \times 100.$$

In certain embodiments, cattery identification and queen identification can be introduced as random effects. In certain non-limiting embodiments, additional biomarkers or factors can include sex, growth rate (0-2 days), stillborn in litter, caesarean, gestation length, parity, age of tomcat, age of queen, or combinations thereof In some non-limiting embodiments, other biomarkers, such as low birth weight, low early growth rate, or combinations thereof can help indicate a higher risk of neonatal mortality.

In certain embodiments, the neonatal mortality application server 112 can analyze the selection 404 of the at least one biomarker. A reference database can be subsequently utilized to analyze the one or more biomarker input(s). In certain embodiments, the reference database can include information or data related to various animals, breeds, species, or any other characteristic of an animal. The reference database can utilize evidenced-based neonatal mortality charts for animals, derived from biomarkers such as birth weight, among others, to create applicable standards, for example, specific to a particular breed.

After analyzing the one or more biomarkers, the neonatal mortality application server 112 can select relevant health trend information of the animal based on selection 404 of the one or more biomarker inputs, and/or display the relevant health trend information as an output 408 relating to the animal on the graphical user interface 400. The relevant health trend information relating to the animal can include determining characteristics and/or comparisons between the biomarker input and data in the reference database relating to similar animals. The output 408 assessment can be generated which, in some embodiments, can display the animal's overall risk for neonatal mortality within the main window 402 of the user interface 400. In certain embodiments, the output 408 assessment can include charts, graphs, graphics, messages, text, icons, or the like.

In certain embodiments, the neonatal mortality application 112 can compare the biometric input and all previously utilized biometric inputs of the particular animal to the reference database. As such, reference curves appropriate to the given inputs can be selected, each of which can be unique or customized to a specific animal. Those reference curves can then be visually displayed on the user interface 400.

In some embodiments, an intervention can be recommended based on any one of the selection 404 of the one or more biomarkers inputs, user requests, and/or the output 408 generated.

In certain embodiments, the neonatal mortality application server 112 can store and/or monitor multiple animals and can further be accessed by multiple users via multiple devices. The data of each animal and a corresponding associated profile can be transferrable to and/or accessed by different users through the neonatal mortality application on any device or system interface. A specific animal can be identified by a unique identification tag, code, picture, number, or the like, which a user can input to retrieve the specific animal's profile and/or history.

In another embodiment, information attained by the neonatal mortality application server 112 can be added to the reference database in order to update the reference database discretely or in real time. The discrete or real time data can be used in clinical or veterinary studies to provide guidance. As such, the use of historical data can preempt the need for future intervention by adjusting and self-updating the system.

In yet another embodiment, neonatal mortality application server 112 can be used to monitor risk factors for neonatal mortality, and the effectiveness of recommended interventions. By tracking when an intervention recommendation is made, the timeline of how quickly (e.g., in days, weeks, months) the animal's health returns to the predetermined range can be monitored. Because neonatal mortality application server 112 can store past animal data within an animal profile, intervention effectiveness can also be monitored.

The present disclosure provides for methods for diagnosing a risk of neonatal mortality in animals, according to certain embodiments described herein. The method generally relates to embodiments, where information is received and displayed on a graphical user interface. In certain embodiments, a user can input data, for example, an animal specific biomarker, and subsequently receive information, data, recommendations, and/or intervention steps relating to the animal's health such as risk for neonatal mortality or similar feature based on an analysis and comparison of the biomarker to a reference database. The application can allow for customization of the biomarkers, information, data, and/or general inputs relating to a wide variety of animals, while maintaining a display of subsequent information, data, recommendations, intervention steps, and/or other outputs relating to the monitoring and/or evaluation of the animal to allow for reduced risk of neonatal mortality. In certain embodiments, one or more biomarker inputs of a first animal can be received. In some embodiments, the first animal can be a non-human animal such as a kitten. The one or more biomarker inputs of the first animal can include animal identification or approximate animal breed, birth weight, and/or early growth rate, among other factors. As discussed above, a user can be prompted to enter the biomarker input relating to the first animal.

In certain non-limiting embodiments, the one or more biomarker inputs of the first animal can be analyzed. In certain embodiments, the analyzing can include comparing the one or more biomarker inputs of the first animal to a reference database. The comparison can obtain health trend information relating to the first animal. The reference database can include biomarker inputs related to animals of the same species as the first animal. The reference database can store values of given biomarkers that are ideal or optimal according to various centiles for a specific type of animal. In some embodiments, the comparison can also calculate the difference between the biomarker input versus a similar, comparable, or related value from the reference database.

In certain non-limiting embodiments, relevant health trend information of the first animal can be selected based on the one or more biomarker input. The health trend information can include informational charts, graphs or curves and/or recommended intervention steps relating to the first animal.

In certain non-limiting embodiments, the relevant health trend information relating to the first animal can be displayed on a graphical user interface.

In certain non-limiting embodiments, an alert can be provided. In certain embodiments, if the alert or recommendation provides a “healthy” status, such alert or recommendation can be directly sent to display on the graphical user interface. However, if the alert or recommendation provides an “unhealthy” status, then the alert or recommendation can further provide an intervention recommendation to be displayed on the graphical user interface. The intervention recommendation can provide one or a plurality of recommended intervention steps.

The present disclosure further provides alternate methods for diagnosing a risk of neonatal mortality in animals, according to one embodiment described herein. The method specifically relates to receiving of specific biomarker information and subsequently identifying a specific subgroup of individual animal(s) who are at risk for neonatal mortality indications, and further receiving information, data, and customized recommendations and/or intervention steps for the specific at risk animal relating to the animal's health, neonatal mortality indication, or similar feature based on an analysis and determination of the biomarker as compared to a reference database. In certain non-limiting embodiments, one or more first biomarker inputs relating to a first animal are received. In some embodiments, the first animal can be a non-human animal such as a kitten. The one or more biomarker inputs of the first animal can include animal identification or approximate animal breed, birth weight, and/or early growth rate, among other factors. As discussed above, a user can be prompted to enter the biomarker input relating to the first animal.

In certain non-limiting embodiments, the one or more first biomarker inputs of the first animal can be compared to at least one predetermined reference biomarker input stored in a reference database. The comparing can obtain relevant health trend information relating to the first animal. The predetermined reference biomarker input can include, for example, related biomarker inputs of normal, healthy animals of the same species as the first animal.

The comparison can obtain health trend information relating to the first animal. The reference database includes biomarker can input related to animals of the same species as the first animal. The reference database can store values of given biomarkers that are ideal, optimal, or preferred according to various centiles for a specific type of animal.

In certain non-limiting embodiments, a determination can be made, based on the comparing, as to whether the first animal is at risk for at least one neonatal mortality indication. In some embodiments, the determining can include calculating a recommended range of the neonatal mortality indication of the first animal. In certain embodiments, an at risk first animal can be diagnosed with the at least one neonatal mortality indication, among other suitable diagnoses.

In certain non-limiting embodiments, a subject determined to be at risk for at least one neonatal mortality indication can be provided with a customized recommendation. The first animal can maintain an increased likelihood of neonatal mortality if the first biomarker input can be determined to be above or below the predetermined reference biomarker input.

In certain non-limiting embodiments, the customized recommendation and the relevant health trend information of the first animal can be displayed on a graphical user interface. The customized recommendation can be an intervention step for correction of the neonatal mortality indication. As such, the customized recommendation can provide one or a plurality of recommended intervention steps for the specific, identified at risk animal(s). In certain embodiments, the recommendation and/or intervention step for the specific, identified at risk animal(s) can include the feeding the animal a special diet.

### 6. EXAMPLES

The presently disclosed subject matter will be better understood by reference to the following Examples, which are provided as exemplary of the disclosure, and not by way of limitation.

### Example 1

### Determination of Predictive Factors of Neonatal Mortality in Kittens

The present Example provides for the determination of predictive factors for indication of neonatal mortality in kittens. A total of 4,152 live-born kittens from 13 breeds, 1,106 litters and 136 French catteries were used. The sex ratio was 1.2 (1,795 males to 1,560 females). Parameters evaluated for each breed or breed group included birth weight, litter size, early growth rate, and litter heterogeneity. The rank for each breed or breed group is provided in Table 1.

TABLE 1
Rank Per Breed For All Parameters Evaluated

| | Ranks | | | | |
|---|---|---|---|---|---|
| | Birth | | Early | Litter | |
| | Weight | Litter | Growth | Hetero- | |
| Breeds/Breed Groups | (g) | Size | Rate | geneity | Sum |
| Abyssinian/Somali | 6 | 12 | 1 | 6 | 25 |
| Balinese/Mandarin/ | 8 | 4 | 12 | 10 | 34 |
| Oriental/Siamese | | | | | |
| Bengal | 12 | 5 | 2 | 1 | 20 |
| Birman | 9 | 11 | 13 | 9 | 42 |
| British | 5 | 6 | 5 | 5 | 21 |
| Chartreux | 2 | 10 | 6 | 11 | 29 |
| Egyptian Mau | 11 | 7 | 9 | 2 | 29 |
| Maine Coon | 1 | 3 | 7 | 7 | 18 |
| Norwegian Forest Cat | 3 | 8 | 11 | 12 | 34 |
| Persian/Exotic | 13 | 13 | 10 | 8 | 44 |
| Ragdoll | 4 | 2 | 4 | 4 | 14 |
| Russian Blue/Nebelung | 10 | 9 | 3 | 13 | 35 |
| Siberian | 7 | 1 | 8 | 3 | 19 |

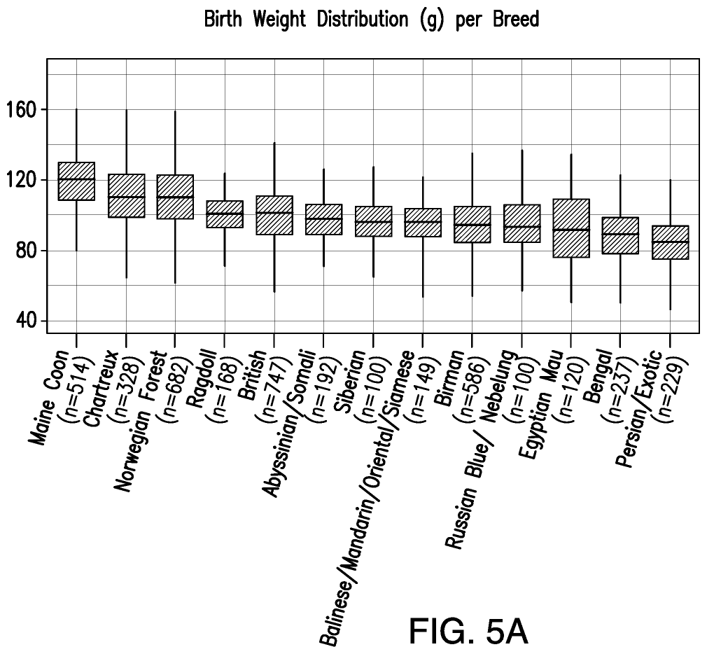

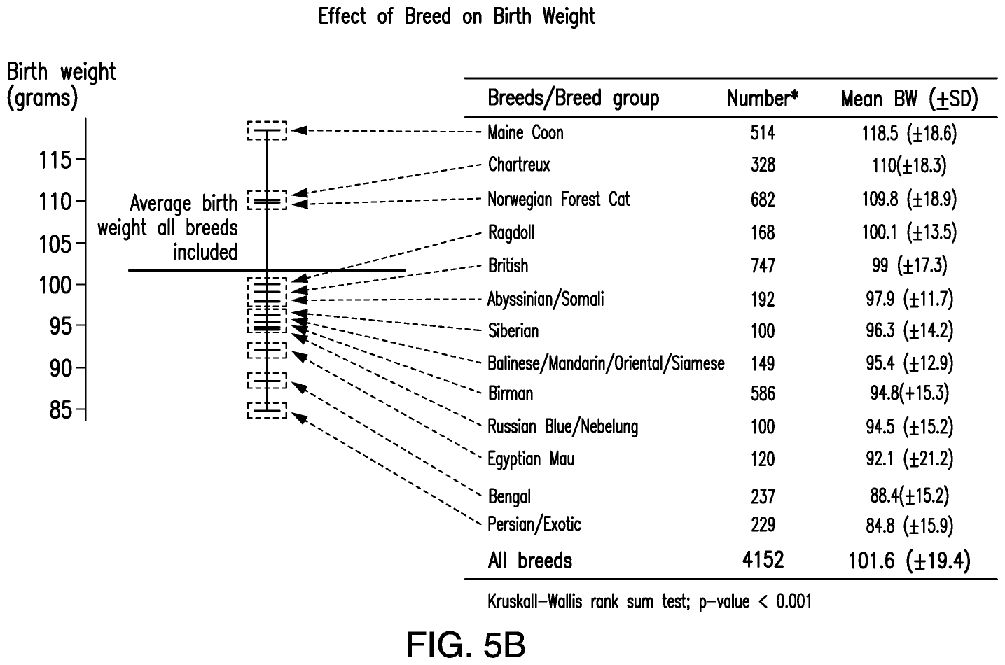

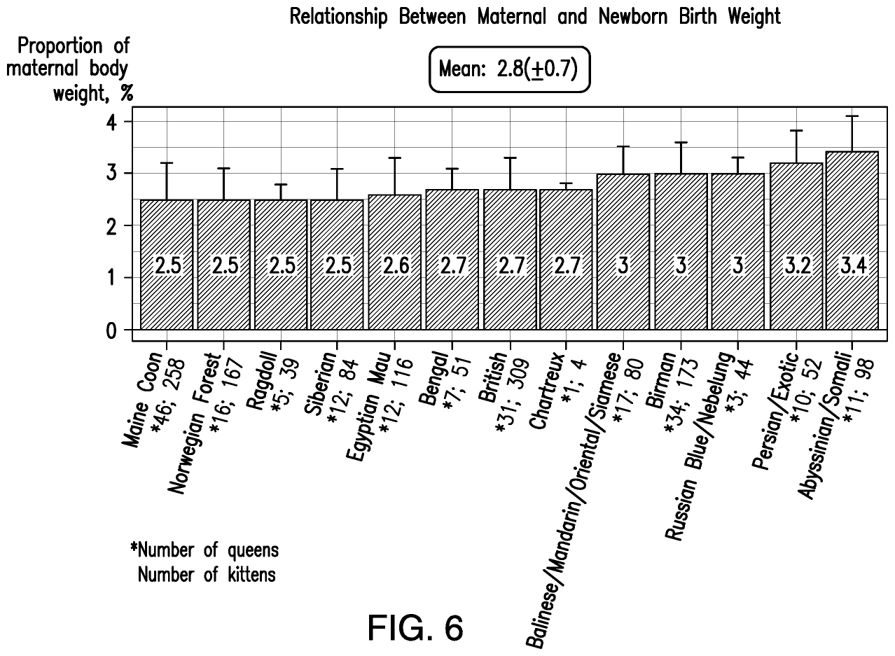

With respect to birth weight, the birth weight distribution per breed is provided in FIG. 5A. The effect of breed on birth weight is provided in FIG. 5B. The relationship between maternal and newborn birth weight per breed is provided in FIG. 6.

Further, generalized mixed models were fitted to determine factors affecting mortality during two different periods: 0-2 days and 2 days-2 months.

The fixed-effects introduced in the models were: birth weight, litter size (total number of kittens born alive), litter heterogeneity (within-litter variation of birth weight, expressed as the coefficient of variation CV, ratio of the standard deviation to the mean), season of queening and early growth rate. The early growth rate can be calculated using the following equation:

$$\frac{weight at 2 days - weight at birth}{weight at birth} \times 100 ,$$

for the period 2 days to 2 months. Cattery and queen were introduced as random effects to deal with the non-independence of kittens sharing the same cattery and the same mother.

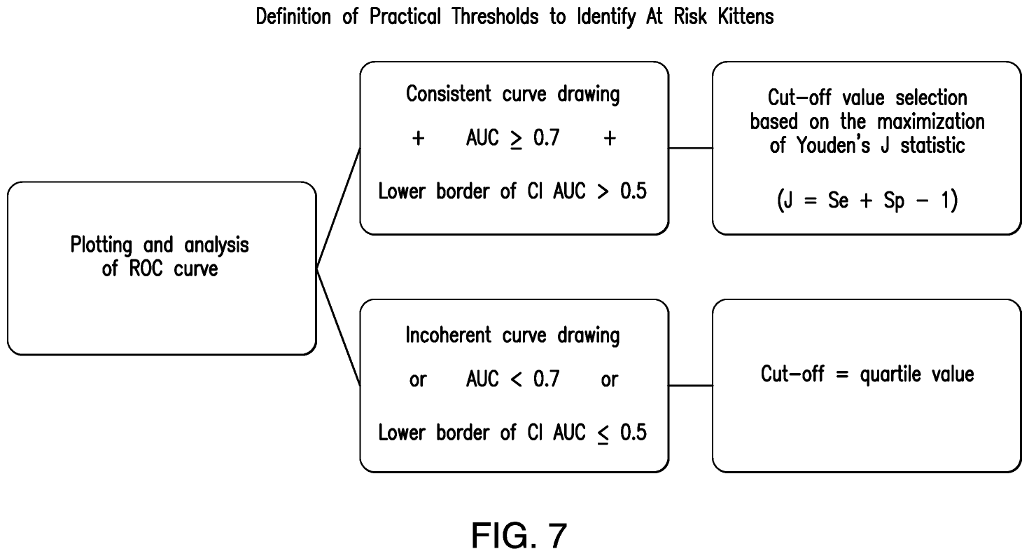

In certain non-limiting embodiments, receiver operating characteristic (ROC) curves were used to identify optimal cut-off values for birth weight regarding mortality during the first two months of life specifically for each breed included. Areas under the ROC curves (AUC) were calculated to estimate the ability of birth weight to discriminate between kittens of different status, such as, dead or alive at two months of life. A flowchart of the definition of practical thresholds to identify at-risk kittens is provided in FIG. 7. As illustrated in FIG. 7, the effectiveness of the parameter to discriminate between kittens dying during neonatal period and those that survive can be assessed using the AUC and its 95% confidence interval (CI). If the AUC is greater than or equal to 0.70 and the lower border of the CI upper is greater than or equal to 0.5, the cut-off value can be determined for each breed on a maximized Youden's J statistic, where J=Sensitivity (Se)+Specificity (Sp)−1. On the other hand, if the AUC is less than 0.70 and the lower border of the CI upper is less than 0.5, the first quartile birth weight value can be used as a threshold. The unvariable analysis can be provided in Table 2. All variables used in the statistical analysis with categorization and missing values can be provided in Table 3.

TABLE 2
Unvariable Analysis

| | Dead | Dead | |
|---|---|---|---|
| | (0-2 months) | (2 days-2 months) | |
| Breed/Breed | <0.001 | <0.001 | Fisher's Exact |
| Group | | | Test |
| Birth Weight | <0.001 | <0.001 | Chi Square Test |
| Stillborn In | 0.656 | 1 | Chi Square Test |
| the Litter | | | |
| Season of | <0.001 | 0.001 | Chi Square Test |
| Whelping | | | |
| Birth Weight | <0.001 | 0.013 | Chi Square Test |
| CV | | | |
| Sex of Kitten | 0.625 | 0.682 | Chi Square Test |
| Litter Size | <0.001 | <0.001 | Chi Square Test |
| Growth Rate | ////// | <0.001 | Chi Square Test |
| 0-2 Days | | | |

*Keeping Parameters With Univariate p-value < 0.20

TABLE 3
Missing Values and All Variables Used in
Statistical Analysis With Categorization

| | | | | | Inclusion |
|---|---|---|---|---|---|
| | | | | Inclusion | in Models |
| | | | | in Models | (2 days- |
| | Regis- | Missing | Catego- | (0-2 months | 2 months |
| Parameter | tered | Values | rization | mortality) | mortality) |
| Breed* | ✓ | 0% | Qualitative | ✓ | ✓ |
| Sex of Kitten | ✓ | 19% | | | |
| Birth Weight | ✓ | 0% | Qualitative | ✓ | ✓ |
| | | | (quartiles | | |
| | | | by breed, | | |
| | | | 4 groups) | | |
| Growth Rate | ✓ | 18% | Qualitative | | ✓ |
| 0-2 Days | | | (quartiles | | |
| | | | by breed, | | |
| | | | 4 groups) | | |
| Litter Size | ✓ | 30% | Qualitative | ✓ | ✓ |
| | | | (quartiles | | |
| | | | by breed, | | |
| | | | 3 groups) | | |
| Stillborn | ✓ | 32% | | | |
| in Litter | | | | | |
| Birth | ✓ | 5% | Qualitative | ✓ | ✓ |
| Weight CV | | | (quartiles | | |
| | | | by breed, | | |
| | | | 3 groups) | | |
| Caesarian | ✓ | 66% | | | |
| Season of | ✓ | 7% | Qualitative | ✓ | ✓ |
| Queening | | | | | |
| Gestation | ✓ | 62% | | | |
| Length | | | | | |
| Parity | ✓ | 71% | | | |
| Age of | ✓ | 75% | | | |
| Tomcat | | | | | |
| Age of | ✓ | 65% | | | |
| Queen | | | | | |
| Cattery | ✓ | 5% | Qualitative | ✓ | ✓ |
| Queen | ✓ | 22% | Qualitative | ✓ | ✓ |

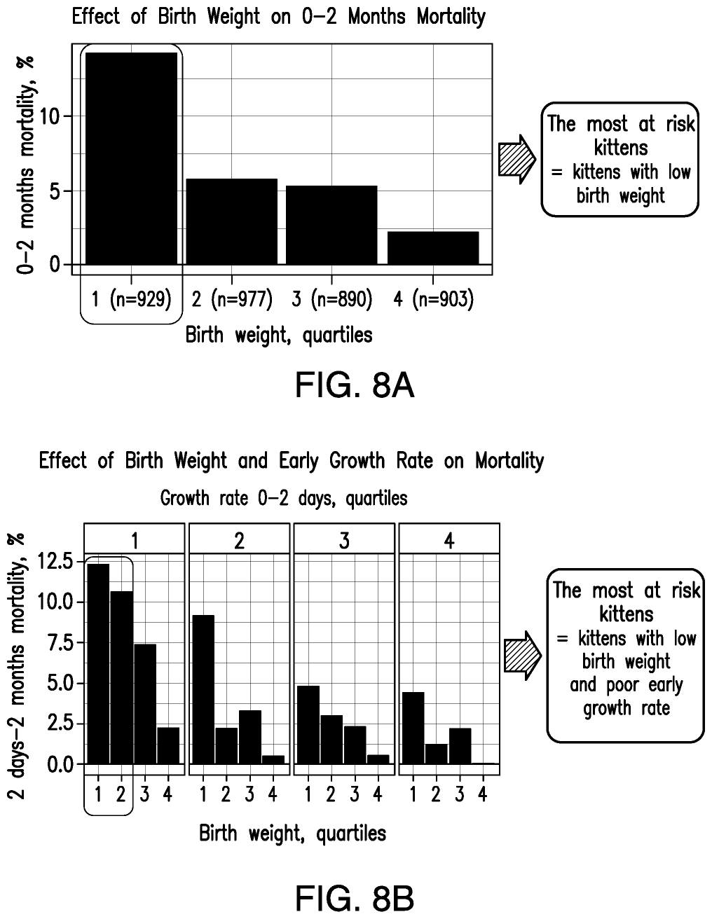

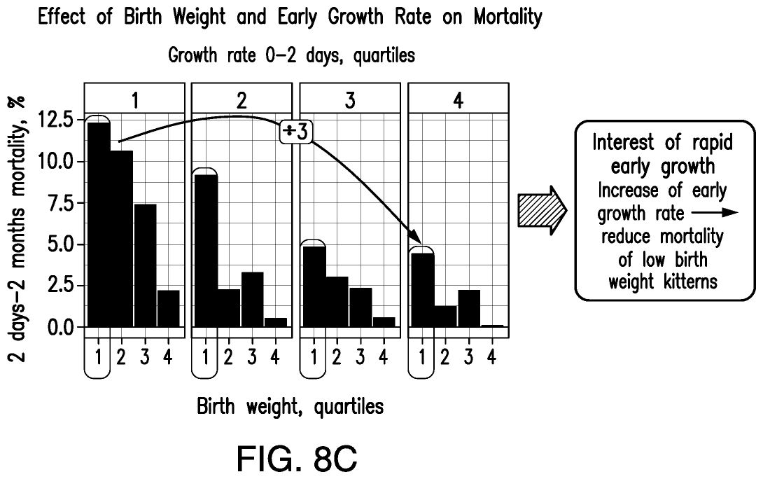

The results of the analysis are provided in FIGS. 8A-8C. FIG. 8A provides the effect of birth weight on mortality within 0-2 months. FIGS. 8B and 8C provide the effect of birth weight and early growth rate on mortality within 2 days to 2 months. In summary, as shown in FIG. 8A, the most at-risk kittens were kittens with low birth weight. As shown in FIG. 8B, the most at-risk kittens were kittens with low birth rate and poor early growth rate. As shown in FIG. 8C, the increase of early growth rate provided a reduced mortality of low birth weight kittens.

A total of 6.8% (95% confidence interval, 95% CI: 6-7.7) of live-born kittens died during the first two months after birth with significant variations between breeds (from 0% in Ragdoll to 15% in Russian Blue).

From all parameters evaluated between 0-2 days, only birth weight was significantly associated with mortality (P<0.001). Mortality was significantly higher in kittens with birth weight lower than the first quartile: 14.2% (95% CI: 12.3-16.6) vs. 4.4% (3.6-5.2). Mortality between 2 days and 2 months of life was significantly influenced by birth weight and early growth rate (both P<0.001). During this period, mortality was significantly higher in kittens with birth weight lower than the first quartile: 8% (95% CI: 6.3-10) vs. 3% (95% CI: 2.4-3.7). Kittens with low birth weight and poor early growth rate (both parameters lower than the first quartile) were at higher risk of death between 2 days and 2 months after birth compared to kittens from other categories: mortality rate respectively at 13.3% (95% CI: 9-18.7) and 3.8% (95% CI: 3.1-4.6). Birth weight critical thresholds have been established in seven (7) breeds (for which AUC≥0.7): Abyssinian/Somali, 95g; British group, 103g; Chartreux, 107g; Egyptian Mau, 85g; Maine Coon, 120g; Oriental group, 77g; Russian Blue, 95g. Two breeds can have a similar birth weight distribution but significant critical thresholds (Oriental group vs. Abyssinian/Somali, for example).

Birth weight critical thresholds presented in this Example, established in seven (7) breeds, would allow the identification of kittens with higher risk of mortality in order to provide them with appropriate nursing and medical care.

Although the presently disclosed subject matter and its advantages have been described in detail, it should be understood that various changes, substitutions and alterations can be made herein without departing from the spirit and scope of the application as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein can be utilized according to the presently disclosed subject matter. Accordingly, the appended claims are intended to include within their scope such processes, machines, manufacture, compositions of matter, means, methods, or steps.

While the foregoing is directed to embodiments described herein, other and further embodiments can be devised without departing from the basic scope thereof. For example, aspects of the present disclosure can be implemented in hardware or software or in a combination of hardware and software. One embodiment described herein can be implemented as a program product for use within a computer system. The program(s) of the program product define functions of the embodiments (including methods described herein) and can be contained on a variety of computer-readable storage media. Illustrative computer-readable storage media include, but are not limited to: (i) non-writable storage media (for example, read-only memory devices within a computer such as CD-ROM disks readable by a CD-ROM drive, flash memory, ROM chips or any type of solid-state non-volatile semiconductor memory) on which information is permanently stored; and (ii) writable storage media (for example, floppy disks within a diskette drive or hard-disk drive or any type of solid-state random-access semiconductor memory) on which alterable information is stored. Such computer-readable storage media, when carrying computer-readable instructions that direct the functions of the disclosed embodiments, are embodiments of the present disclosure.

For any patents, patent applications, publications, product descriptions, and protocols are cited throughout this application, the disclosures of all of which are incorporated herein by reference in their entireties for all purposes.

1. A method of diagnosing a risk of neonatal mortality in non-human animals, comprising:
receiving one or more first biomarker inputs relating to a first animal;
comparing the one or more first biomarker inputs of the first animal to at least one predetermined reference biomarker input stored in a reference database, wherein the predetermined reference biomarker input includes related biomarker inputs of animals of a same species;
determining, based on the comparing, whether the first animal is at risk for at least one neonatal mortality indications;
providing the non-human animals determined to be at risk for the at least one neonatal mortality indications with a customized recommendation, wherein the first biomarker input determined to be above or below the predetermined reference biomarker input indicates an increased likelihood of neonatal mortality in the first animal; and
displaying the customized recommendation and relevant health trend information of the first animal on a graphical user interface.

2. The method according to claim 1, wherein the customized recommendation is an intervention step for correction of the at least one neonatal mortality indications.

3. The method according to claim 1, wherein the one or more first biomarker inputs of the first animal comprise animal breed, birth weight, litter size, litter heterogeneity, queening season, or early growth rate.

4. The method according to claim lany of claims 1, wherein the at least one neonatal mortality indication comprises low birth weight, low early growth rate, or combinations thereof.

5. A computer system for diagnosing a risk of neonatal mortality in non-human animals, comprising:
a processor; and
a memory storing instructions that, when executed by the processor, cause the computer system to:
receive one or more first biomarker inputs relating to a first animal;
compare the one or more first biomarker inputs of the first animal to at least one predetermined reference biomarker input stored in a reference database, wherein the predetermined reference biomarker input includes related biomarker inputs of animals of a same species as the first animal;
determine, based on the comparing, whether the first animal is at risk for at least one neonatal mortality indications;
provide the non-human animals determined to be at risk for the at least one neonatal mortality indications with a customized recommendation, wherein the first biomarker input determined to be above or below the predetermined reference biomarker input indicates an increased likelihood of neonatal mortality in the first animal; and
display the customized recommendation and relevant health trend information of the first animal on a graphical user interface.

6. The system according to claim 5, wherein the customized recommendation is an intervention step for correction of the at least one neonatal mortality indications.

7. The system according to claim 5, wherein the one or more first biomarker inputs of the first animal comprise animal breed, birth weight, litter size, litter heterogeneity, queening season, or early growth rate.

8. The system according to claim 5, wherein the at least one neonatal mortality indication comprises low birth weight, low early growth rate, or combinations thereof.

9. The method according to claim 1, wherein the reference database comprises at least one neonatal mortality chart for animals.

10. The method according to claim 1, further comprising:
adding the relevant health trend information of the first animal to the reference database.

11. The method according to claim 1, further comprising:
generating a profile associated with the one or more first biomarker inputs;
adding the profile to the reference database; and
associating the profile with one or more of an identification tag, a code, a picture, or a number.

12. The method according to claim 1, wherein displaying the customized recommendation and relevant health trend information further comprises:
generating a reference curve related to the one or more first biomarker inputs; and
displaying the reference curve on the graphical user interface.

13. The method according to claim 1, wherein the customized recommendation is be based on one or more of the first one or more biomarker inputs, one or more user requests, and the risk for the at least one neonatal mortality indications.

14. The method according to claim 1, wherein comparing the one or more first biomarker inputs of the first animal to at least one predetermined reference biomarker input stored in a reference database further comprises:
calculating a difference between the first one or more biomarker inputs with a related value from the reference database.

15. The system of claim 5, wherein the reference database comprises at least one neonatal mortality chart for animals.

16. The system of claim 5, wherein the instructions further cause the computer system to:
add the relevant health trend information of the first animal to the reference database.

17. The system of claim 5, wherein the instructions further cause the computer system to:
generate a profile associated with the one or more first biomarker inputs;
add the profile to the reference database; and
associate the profile with one or more of an identification tag, a code, a picture, or a number.

18. The system of claim 5, wherein the instructions further cause the computer system to:
generate a reference curve related to the one or more first biomarker inputs; and
display the reference curve on the graphical user interface.

19. The system according to claim 5, wherein the customized recommendation is be based on one or more of the first one or more biomarker inputs, one or more user requests, and the risk for the at least one neonatal mortality indications.

20. The system according to claim 5, wherein the instructions further cause the computer system to:
calculate a difference between the first one or more biomarker inputs with a related value from the reference database.
